(19) United States
(12) Patent Application Publication
(10) Pub. No.: US 2012/0259365 A1
(43) Pub. Date: Oct. 11, 2012

(54) FACET FUSION IMPLANT, INSTRUMENTATION, AND SURGICAL PROCEDURE

(75) Inventor: Marc E. Richelsoph, Charlotte, NC (US)

(73) Assignee: Intelligent Implant Systems, Charlotte, NC (US)

(21) Appl. No.: 13/269,056

(22) Filed: Oct. 7, 2011

Related U.S. Application Data
(60) Provisional application No. 61/472,274, filed on Apr. 6, 2011.

(51) Int. Cl. A61B 17/70; A61B 17/56; A61B 17/16; A61B 17/88

(52) U.S. Cl. 606/247; 606/279; 606/86 A; 606/80

(57) ABSTRACT

A facet fusion implant, including a body having stepped surface means for contacting a stepped surface of a facet joint and preventing the facet fusion implant from coming out of the facet joint. An external sleeve compacting instrument for inserting bone enhancing materials in an implant. A method of using an external sleeve compacting instrument. A method of preparing a facet fusion implant with bone enhancing materials. A method of creating a facet joint fusion by inserting the implant in a facet joint, contacting the stepped surface with a stepped surface of the facet joint, preventing motion of the implant within the facet joint, and creating a facet joint fusion. A method of preventing motion of an implant within a facet joint. A guide instrument and a reamer for inserting an implant in a facet joint. A method of inserting an implant into a facet joint.

### BACKGROUND OF THE INVENTION

1. Technical Field

The present invention relates to a facet fusion implant. More specifically, the present invention relates to a load sharing facet fusion implant that assists in fusing two vertebrae by providing the benefits of a spacer with the ability to allow load sharing with the bone graft to provide better fusion results.

2. Background Art

Facet joints in the body provide stability to the spine while allowing controlled motion under normal physiological loads. However, like all other joints in the body, they are subject to degeneration over time. Severe enough degeneration often causes patients pain, and must be treated. More recently, specially shaped bone implants have been used in order to place bone directly into the facet joint space to create better immediate stability with better long term fusion. Typically, these implants are constructed from allograft. In addition, by using bone, a natural scaffold is created to allow the facet joint to fuse. Other devices that fuse facet joints are screws or metallic implants that do not go into the facet joint, but rather through both sides of the joint in order to securely fasten the faces of the joint together to prevent motion. These screw or cross joint devices are not the focus of the novel invention disclosed herewith.

The main problem with these conventional facet joint fusion devices, or the bone implants that are placed within the joint, is that the implants have a tendency to back out over time and not stay within the prepared facet joint space. It is well understood that the spine has six degrees of freedom and motion of the spine is constant. The goal of fusion is to significantly reduce or stop motion at the painful level, but fusion takes time. Therefore, the implant must be stable for the time until fusion can occur. Flexion—extension of the spine or anterior—posterior bending, such as leaning forward or backwards places significant strain on the spine and therefore motion. Bending forward opens the facet joints and creates a force that naturally faces posteriorly. This load therefore wants to force any implant within the facet joint posteriorly and out of the joint. This is a clinical issue and has been observed with the current designs.

A number of these implants are smooth, cylindrical, and have no means to hold them in place within the facet joint. They rely only on compressive force to hold them. For example, U.S. Pat. No. 7,708,761 to Petersen discloses a bone plug in a smooth cylindrical shape with a vertical central channel for the insertion of material for assisting in fusing the bone plug in place in a spinal joint. Other cylindrical implants exist with grooves, such as NUFIX™ (NuFix). There are some threaded implants, however, a bone thread must be tapped and this thread is thin. Facet Fusion Implants are small and generally less than 10 mm in length, which does not provide much room for a good thread. The threads do not provide any advantage but instead weaken the allograft. Current implants do not have any resistance to expulsion and rely on a press fit with the prepared hole in the facet joint.

While these prior devices can be suitable for limited usage to which they somewhat address, they are not as suitable to providing a stable device the permits load sharing with the facet joint while providing sufficiently rigid support of the spine during the healing process.

In these respects, the facet fusion implant according to the present invention substantially departs from the conventional concepts and designs of the prior art, and in so doing provides an apparatus primarily developed for the purpose of creating facet joint fusions while providing sufficiently rigid support of the spine while preventing expulsion of the fusion device under normal anatomical loads.

### SUMMARY OF THE INVENTION

The present invention provides for a facet fusion implant, including a body having a stepped surface for contacting a stepped surface of a facet joint and preventing the facet fusion implant from coming out of the facet joint.

The present invention provides for a facet fusion implant, including a body having a stepped surface for contacting a stepped surface of a facet joint and preventing the facet fusion implant from coming out of the facet joint, the body also including grooves for increasing surface grip and carrying bone enhancing materials.

The present invention also provides for a facet fusion implant, including a body having a stepped surface for contacting a stepped surface of a facet joint and preventing the facet fusion implant from coming out of the facet joint, the body including an internal bore for carrying bone enhancing materials.

The present invention provides for a facet fusion implant, including a body having a stepped surface for contacting a stepped surface of a facet joint and preventing the facet fusion implant from coming out of the facet joint, the body including an internal bore for carrying bone enhancing materials, and at least one outer hole for flowing blood and bone enhancing materials to the internal bore.

The present invention further provides for an external sleeve compacting instrument for inserting bone enhancing materials in an implant, including a tube having at least one flexible portion and an opening section that expands outward.

The present invention provides for a method of using an external sleeve compacting instrument, by inserting bone enhancing materials through an opening section that expands outward, and into a tube, pushing an implant through the opening section and into the tube, and forcing the bone enhancing material into a structure of the implant chosen from the group consisting of grooves, internal bores, outer holes, rough body surfaces, and combinations thereof by a flexible portion of the tube.

The present invention also provides for a method of preparing a facet fusion implant with bone enhancing materials, by inserting bone enhancing material into an internal bore of a body of the implant, the body including stepped surface means for contacting a stepped surface of a facet joint and preventing said facet fusion implant from coming out of said facet joint.

The present invention provides for a method of creating a facet joint fusion, by inserting an implant in a facet joint, the implant including a body having a stepped surface, contacting the stepped surface with a stepped surface of the facet joint, preventing motion of the implant within the facet joint, and creating a facet joint fusion.

The present invention further provides for a method of preventing motion of an implant within a facet joint, by contacting a stepped surface of the implant with a stepped surface of the facet joint, and preventing motion of the implant within the facet joint.

The present invention provides for a guide instrument for inserting an implant in a facet joint, including a handle with an enlarged proximal end, a tubular portion operably connected to the handle, the tubular portion including a distal end having a bone engagement mechanism for attaching to bone surrounding the facet joint.

The present invention provides for a reamer for preparing a cavity and step in a facet joint, including a shaft having a proximal end, the proximal end having a stop mechanism for stopping against a guide instrument and an interface mechanism for interfacing with a handle, and a distal end including a cutting mechanism for cutting bone in the facet joint.

The present invention also provides for a method of inserting an implant into a facet joint, by locating the center of the facet joint and the relative angle with a facet locator, inserting the guide instrument over the facet locator until the distal end contacts the bone around the facet joint, removing the facet locator, drilling a hole that matches a first diameter of the implant, inserting the reamer until the stop mechanism engages the enlarged proximal end of the guide instrument, cutting a pocket with the reamer within the facet joint bone matching a second diameter of the implant, removing the reamer, inserting the implant in the facet joint, and removing the guide instrument.

The present invention further provides for a method of inserting an implant into a facet joint, by creating a pocket within the facet joint that is a negative image of the implant, and popping the implant in place within the pocket.

The present invention provides for a facet fusion implant including a body having a locking mechanism for locking the body in a facet joint.

The present invention also provides for a method of locking an implant in a facet joint by applying force to the implant in an anterior direction, applying force to the implant in a posterior direction, and locking the implant in the facet joint.

### DESCRIPTION OF THE DRAWINGS

Other advantages of the present invention are readily appreciated as the same becomes better understood by reference to the following detailed description when considered in connection with the accompanying drawings wherein:

FIG. 1 is an isometric view of one variation of the facet fusion implant;

FIG. 2 is a side view of the facet fusion implant as shown in FIG. 1;

FIG. 3 is a back view of the facet fusion implant as shown in FIG. 1;

FIG. 4 is an isometric view of a variation of the facet fusion implant;

FIG. 5 is a detailed side view of the facet fusion implant shown in FIG. 4;

FIG. 6 is another variation of the facet fusion implant;

FIG. 7 is another variation of the facet fusion implant;

FIG. 8 is a variation of the facet fusion implant shown in FIG. 7;

FIG. 9 is a section view of the facet fusion implant shown in FIG. 7;

FIG. 10 is an exploded view of the facet fusion implant and fusion enhancing material;

FIG. 11 shows a method of loading the facet fusion implant via syringe;

FIG. 12 is a perspective view of an instrument for loading the facet fusion implant;

FIG. 13 is a perspective view of a guide instrument for temporary attachment to the facet joint;

FIG. 14 is a detailed view of the engagement surface of the guide instrument;

FIG. 15 is a perspective view of a reamer;

FIG. 16 is a detailed view of the reamer;

FIGS. 17A-17I are views of steps of the method of inserting the facet fusion implant;

FIGS. 18A-18D are perspective views of the implant being positioned in the facet joint; and

FIGS. 19A-19C are perspective views of alternative body shapes of the implant.

### DETAILED DESCRIPTION OF THE INVENTION

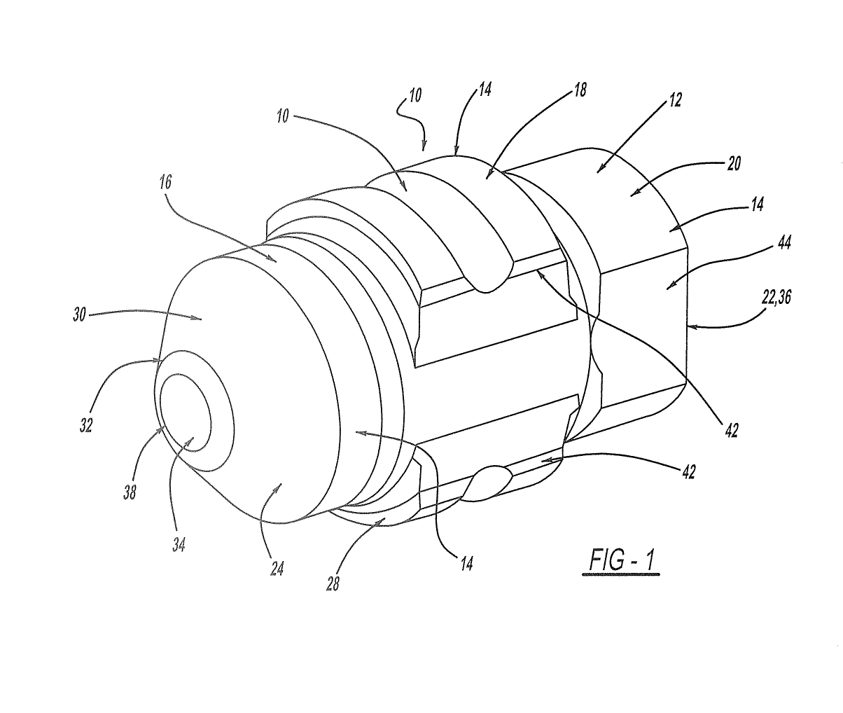

In view of the foregoing disadvantages inherent in the known types of facet fusion devices now present in the prior art, the present invention provides a new facet fusion implant, shown generally at 10 in FIG. 1, that by its novel construction permits the implant 10 to lock into a prepared space within a facet joint to prevent expulsion of the implant 10 while providing sufficiently rigid support of the spine during the healing process. The facet fusion implant 10 generally includes a body 12 having a stepped surface 14, wherein the stepped surface contacts a stepped surface of a facet joint and prevents the implant 10 from coming out of the facet joint.

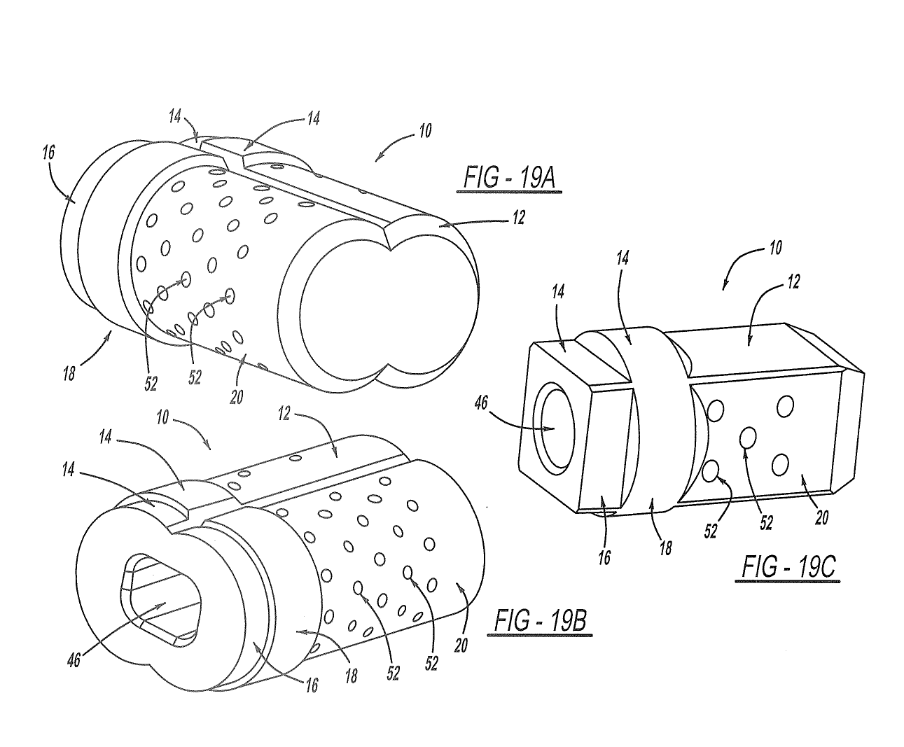

The body 12 is essentially cylindrical in shape. Of course, the body 12 does not need to be cylindrical, but can also be a figure eight shape (FIGS. 19A-19B), square (FIG. 19C), rectangular, or other shapes. The body 12 of facet fusion implant 10 can be made of various materials, such as, but not limited to, autogenous and allograft bone, bone substitutes, polymers such as PEEK, or other porous materials, such as titanium or tantalum meshes. The body 12 can be made of a single material or different materials can be used for different parts. The healing process is one of remodeling, replacement, and resorption of bone. The bone or bone substitute material is not alive, and in the case of autogenous bone, it is not alive either once the bone is taken from its initial location. It acts as a source of calcium, minerals, and structure for the cells to restructure. In a successful fusion whereby the implant fuses within the facet joint, this non-living material is replaced or at least incorporated by living bone. For fusion to occur, the graft, bone substitutes, or osteoinductive material must be held rigidly and without any significant motion. Micromotion often causes non-fusions and must be avoided.

In order to prevent excessive motion in any direction and prevent non-fusions, the stepped surface 14 provides a very important function and prevents the facet fusion implant 10 from coming out of the facet joint. In other words, the implant 10 fits into the facet joint that also has a stepped surface that matches that of the stepped surface 14. When inserted, the stepped surface 14 contacts the stepped surface of the facet joint. Thus, any motion to push the implant 10 out posteriorly is prevented by the engagement of the two stepped surfaces. The goal of the present invention was to create a robust feature to resist posterior expulsion. Furthermore, as the spine moves, the facet joints also move. The stepped surface 14 of the present invention resists this motion. The implant 10 cannot move anteriorly due to drill depth and the stepped surface 14 prevents posterior motion. Thus, forces in either direction create an opposite force to lock the implant 10 and the facet joint together. In other words, the stepped surface 14 in combination with the anterior area of the body 12 adjacent to the prepared drilled facet joint act as a locking mechanism to maintain the position of the implant 10. The implant 10 applies an anterior force to the drilled portion of the facet joint and the stepped surface 14 applies a posterior force in the facet joint.

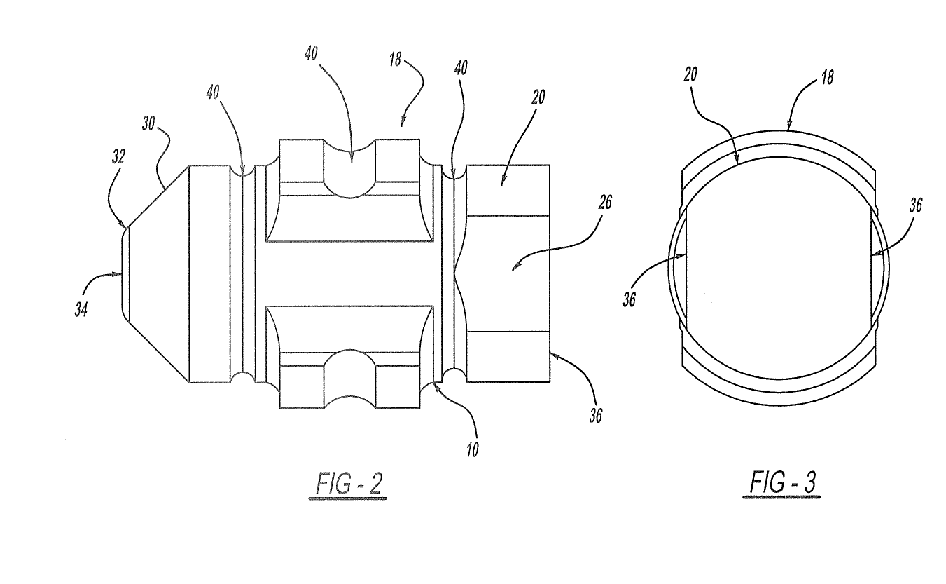

Stepped surface 14 is essentially a raised portion on the body 12. Therefore, the body 12 includes at least two sections, a first section 16 and a second section 18, that are different sizes in diameter and are operably connected. Additional sections can be included as desired, i.e. additional steps. In one example of additional sections, FIGS. 1-3 show a stepped surface 14 with first section 16 and a third section 20 that have smaller diameters than second section 18. The first section 16 and the third section 20 can be the same diameter or can be different diameters as required. The stepped surface 14, and therefore sections 16, 18, and 20, can be different sizes according to the requirements of the patient. Each of the sections 16, 18, 20 of the body 12 match the size of the stepped surface of the facet joint.

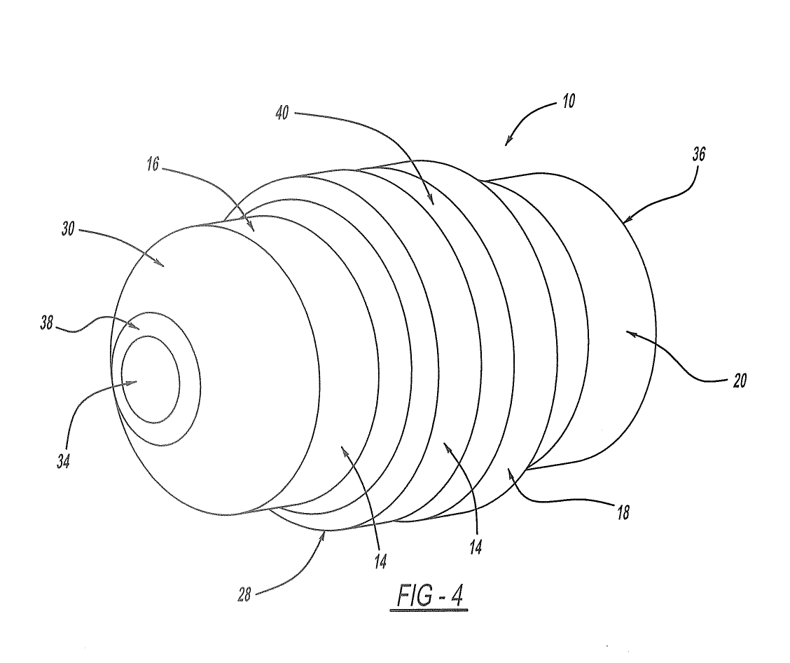

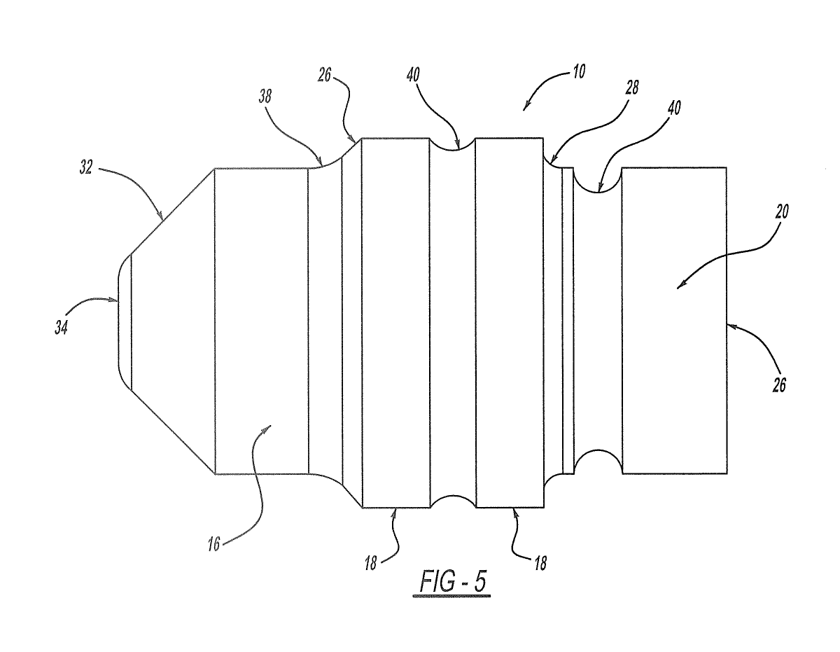

Also, the implant 10 can also have only a portion of the larger diameter in the second section 18, with at least one portion machined away to create a flat surface 42, as shown in FIGS. 1-3. Preferably, two sides of the second section 18 are machined or formed with flat surfaces 42. This effectively creates a bayonet fixation approach, whereby the implant 10 is inserted into a prepared hole and groove such that the groove matches the features of the implant 10. This creates a keyway. When the implant 10 is inserted into the bone, the flat surfaces 42 are lined up with the keyway. After insertion, the implant is rotated 90 degrees or some portion thereof, to effectively lock it in place, similar to a bayonet type of locking. In order to rotate the implant 10 with an instrument, the last stepped surface 14 (i.e. the third section 20 shown in the FIGURES) includes a flat surface 44 that engages with the instrument. Alternatively, the second section 18 can be unbroken by any flat surfaces, as shown in FIGS. 4 and 5.

These implant sections 16, 18, 20 are preferably machined from a single piece of material due to the size of the implant 10. It is also possible for the larger diameter section to be a sleeve that is slipped over a smaller section in order to create the stepped surface 14. In the preferred embodiment, the single machined part is shaped in the form of a cylinder, with a larger cylindrical section 18 in the middle. This larger cylindrical section 18 creates a face that is 90 degrees relative to the smaller diameter on the posterior side 22. The posterior end 22 of the implant 10 (i.e. the end of the last section of the implant 10) can include a flat surface 36 for ease in machining the implant 10. By allowing a larger diameter in the middle of the body 12 and creating a step in the implant 10, the face between the two diameters effectively creates a flat surface 28. On the anterior side 24 of the implant 10, the transition from the smaller diameter of the first section 16 to the larger diameter of the second section 18 is preferably a tapered or chamfered transition 26. In other words, the stepped surface 14 preferably includes a tapered or chamfered transition 26 between steps. Of course, it is possible to leave a small step and not chamfer or taper the entire lead-in face. The primary reason for the taper or chamfer 26 is to allow for easy insertion of the implant 10 into the prepared opening in the facet joint.

The first section 16 is the section that is first inserted into the bone and can also include a tip 30 that includes a chamfer or taper 32 for ease of insertion. The tip 30 can further include a flat tip surface 34, although the tip 30 can also be pointed. In order to provide a smooth transition between the flat tip surface 34 and the chamfer 32, a small blend radius 38 can be formed or machined into the body 12.

Grooves 40 can be included in various places on the body 12 as desired. For example, a central groove 40 can be cut into the surface of the second section 18, as shown in FIGS. 1-5. Additional grooves can also be included on the first section 16 and third section 20 at any suitable place. These grooves 40 can be machined into the body 12 for a few reasons. One reason is that grooves increase surface area in order to increase surface grip with the bone into which the implant 10 is inserted. However, more importantly, they can be used to carry bone enhancing materials such as osteoinductive and osteoconductive materials, as well as other materials further listed below. Therefore, another novel feature of the implant 10 is to provide a design, means, and method for holding fusion enhancing materials as well as the same for packing the materials into the graft.

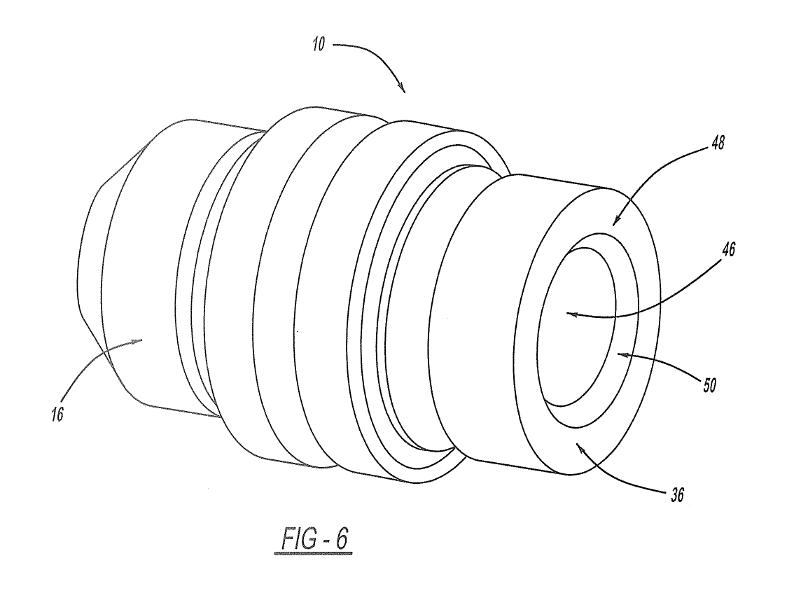

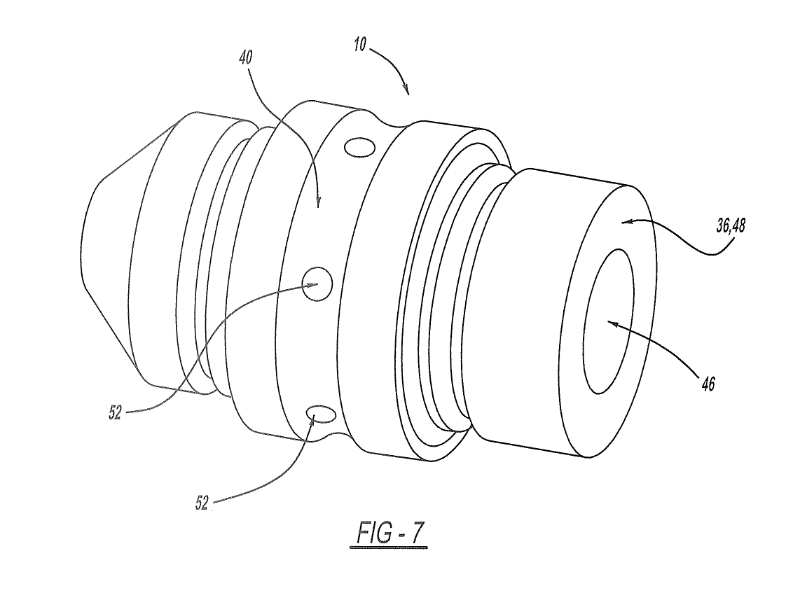

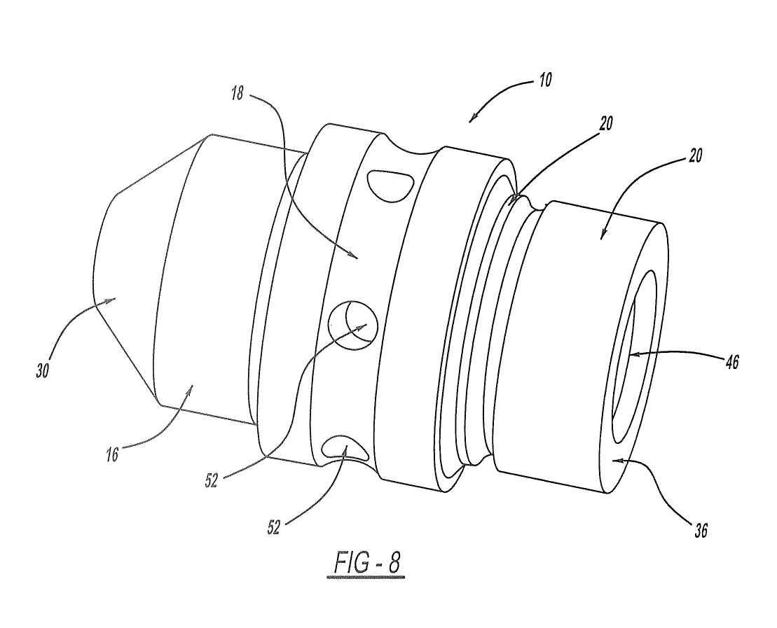

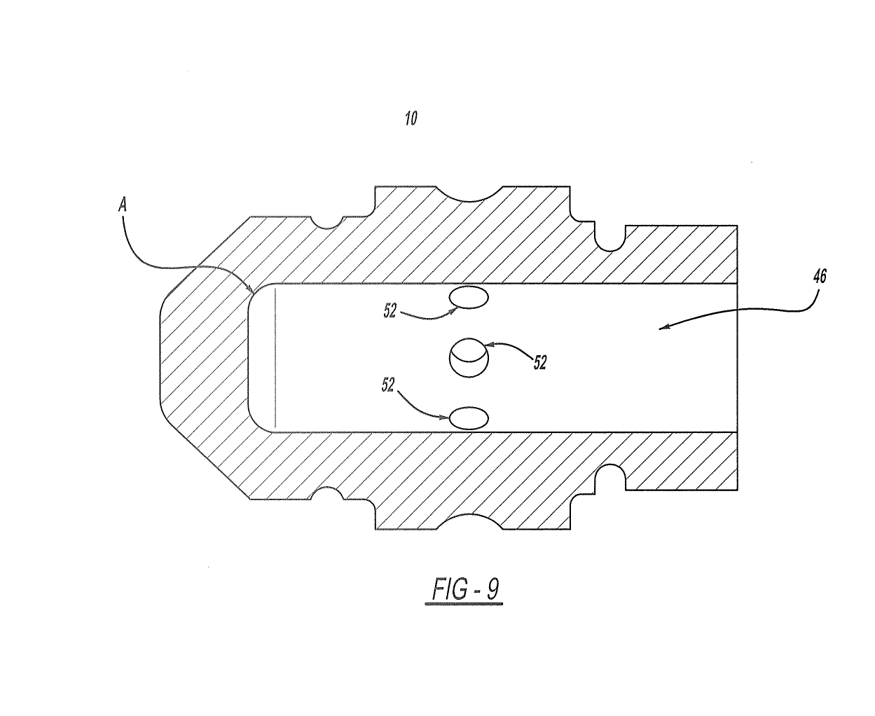

The facet fusion implant 10 can further include an internal bore or hole 46, as shown in FIG. 6. The internal bore 46 can be any diameter desired. The internal bore 46 is preferably partial so as not to go all the way through the body 12 as shown at A in FIG. 9 (i.e. starting at the second (or higher number) section 18 and going towards the first section 16 but not through the first section 16); however, the internal bore 46 can also run the entire length of the body 12. A proximal end 48 (and a distal end if the internal bore 46 runs the length of the body 12) of the internal bore 46 can include a chamfer 50 to allow for easier access to the internal bore 46. The internal bore 46 allows the center of the implant 10 to be packed with bone enhancing materials (osteoinductive or osteoconductive materials), including, but not limited to Demineralized Bone Matrix (DBM), calcium sulfate, BMPs (Bone Morphogenetic Proteins), autograft, or stem cell containing materials or carriers, such as amniotic sheeting or fluid. Demineralized Bone Matrix is a biologic material that is often supplied in a putty form. It can be mixed with other biologic materials, such as bone marrow or autogenous bone to create a better osteoinductive and osteoconductive material than allograft bone. BMPs have the potential to increase fusion rates and can often be supplied as a putty or mixed with DBM to make a putty.

At least one outer hole 52, shown in FIGS. 7-8, can further be included that penetrate the body 12 and connect with the internal bore 46, thereby creating a passageway or multiple passageways for blood and bone enhancing materials and fluids to flow through, i.e. the outer holes 52 are in fluid connection with the internal bore 46. This allows the internal bore 46 to be packed with bone enhancing material and have direct contact with fluids and bone external to the internal bore 46. Preferably, an array of multiple holes 52 are formed or drilled into the implant 10 to maximize access to the internal bore 46. However, size of the holes 52 must be carefully controlled in order to maintain the maximum possible implant strength. Materials that have natural porosity, such as titanium or tantalum foams can effectively have holes 52 already present and therefore do not require a separate drilling or forming process to create them. FIG. 9 is a section view showing that the holes 52 are formed through the body 12 into the internal bore 46. While the holes 52 are shown perpendicular to the internal bore 46, they can also be angled relative to the axial centerline of the implant 10. Obviously, there can also be more than one set of holes 52 in different locations along the implant 10. While FIGS. 7-8 show the holes 52 along the second section 18, it should be understood that the holes 52 can be located anywhere on the body 12.

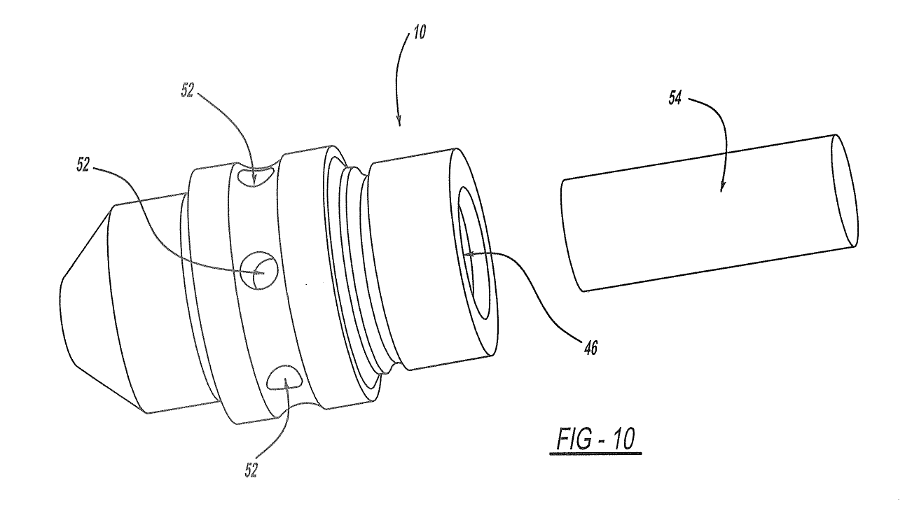

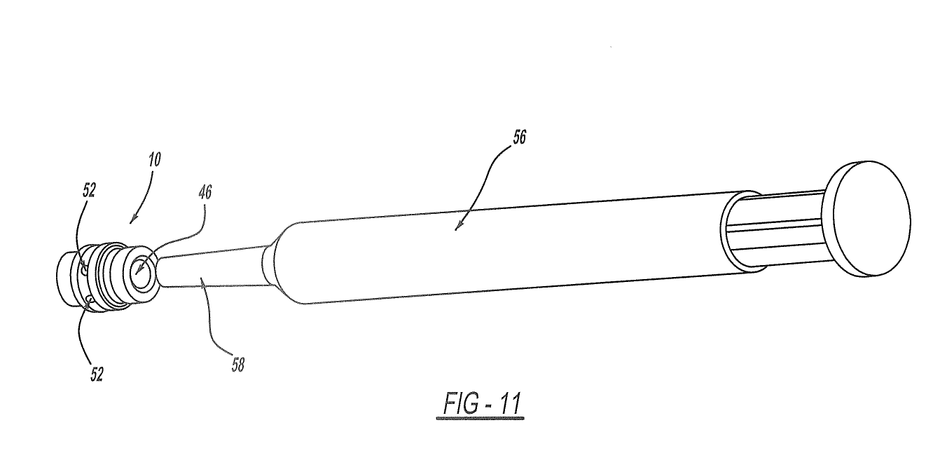

There are several methods of filling the internal bore 46 with the bone enhancing material described above. In one method, the bone enhancing material can optionally be preformed as a plug 54 shown in FIG. 10 and can be inserted within the internal bore 46. The plug 54 is shaped preferably as a cylinder and is of sufficient size to fit within the internal bore 46. Alternatively, the plug 54 can be any shape or any shape to fit securely within the internal bore 46. In addition, some materials that are not normally cylinders can be formed into cylinders for easier insertion. One material that has great potential is amniotic tissue. While normally in sheet form, it can be rolled and inserted into the internal bore 46. Amniotic tissue contains stem cells, which can enhance bone growth and fusion.

Alternatively, a syringe 56 can be used to fill the implant 10 via forcing granular materials, bone chips, or other materials into the internal bore 46 by injection. It is possible by doing so under pressure to actually force the bone enhancing material into the internal bore 46 and into the outer holes 52, effectively filling all the available spaces with the bone enhancing material. The syringe 56 has a tip 58 for engagement with the implant 10. This tip 58 can simply press fit into the internal bore 46 in the implant 10 or engage via a locking feature, such as a bayonet or Luer Lock type fitting.

In another method of packing bone enhancing materials into the implant 10, shown in FIG. 12, bone enhancing materials are inserted into an opening 60 of an external sleeve compacting instrument 62 to a center hole or passageway 64 thereof. Then, the implant 10 is inserted through the opening 60 into the center hole 64, and the bone enhancing materials are forced into the implant 10. Essentially, the external sleeve compacting instrument 62 is a tube in the shape of the implant that has at least one flexible portion 66 and a section that expands outward (the opening 60), such as in the shape of a funnel. The diameter of the opening 60 is larger than the diameter of the implant 10 such that the implant 10 can be guided into the center hole 64 in the external sleeve compacting instrument 62. Bone enhancing material, such as Demineralized Bone Matrix putty, which is demineralized bone mixed with a liquid to create a soft material with the consistency of putty, or any other material described herein, is placed in the opening 60 of the external sleeve compacting instrument 62. As the implant 10 is pushed into the external sleeve compacting instrument 62, the bone enhancing material is forced into the grooves 40, internal bore 46, outer holes 52, and any other rough surface on the body 12 of the implant 10 or combinations of these structures by the flexible portion 66. It is preferable that the flexible portion 66 is made of a material such as silicone or other polymers, to allow it to flex and expand with insertion of the implant 10. It is also preferable that the external sleeve compacting instrument 62 be disposable. While the implant 10 can be inserted and then removed, it is also possible to include an orifice 66 in the opposite end relative to the larger opening 60 such that the implant 10 can be packed and pushed completely through the external sleeve compacting instrument 62. With the orifice 66, it is preferable that it is small enough to maintain the position of the implant 10 within the external sleeve compacting instrument 62 while it is being filled with bone enhancing material, but large enough to be able to remove the implant 10 therefrom. Also, by attaching the implant 10 to an inserter that can also be used within a guide instrument 70 further described below, the surgeon does not need to handle the implant 10 directly nor contact the biological material, but can insert the implant 10 and inserter through the compacting instrument 62. In addition, extra biological material is maintained within the compacting instrument 62 and can be used for additional facet joint implants used at the same surgery.

The facet fusion implant 10 can generally be used in a method of creating a facet joint fusion, by inserting the implant 10 including the body 12 having the stepped surface 14 in a facet joint, contacting the stepped surface 14 with a stepped surface of the facet joint, preventing motion of the implant 10 within the facet joint, and creating a facet joint fusion.

The insertion of the implant 10 can be accomplished by any means known in the art, and several methods are further described below. Prior to insertion, bone enhancing material can be inserted in the implant 10 as described above by either inserting a plug, filling and injecting with a syringe, or using the external sleeve compacting instrument.

The stepped surface 14 contacts the stepped surface machined into facet joint by the reamer. As described above, each section 16, 18, 20 of the body 12 of the implant 10 can be an appropriate diameter in order to line up the stepped surface 14 with the stepped surface of the facet joint.

By matching these surfaces, any posterior and anterior motion of the implant 10 within the facet joint is prevented, allowing fusion to occur. This method has not been performed in the prior art because, as discussed above, prior art implants do not prevent motion from occurring once placed within the facet joint and therefore cannot completely fuse the implant with the facet joint.

The present invention also provides more generally for a method of preventing motion of an implant within a facet joint, by preparing a cavity and stepped surface within the facet joint with a reamer, contacting the stepped surface 14 of the implant 10 with the stepped surface of the facet joint, and preventing motion of the implant 10 within the facet joint. Each of these steps are described above.

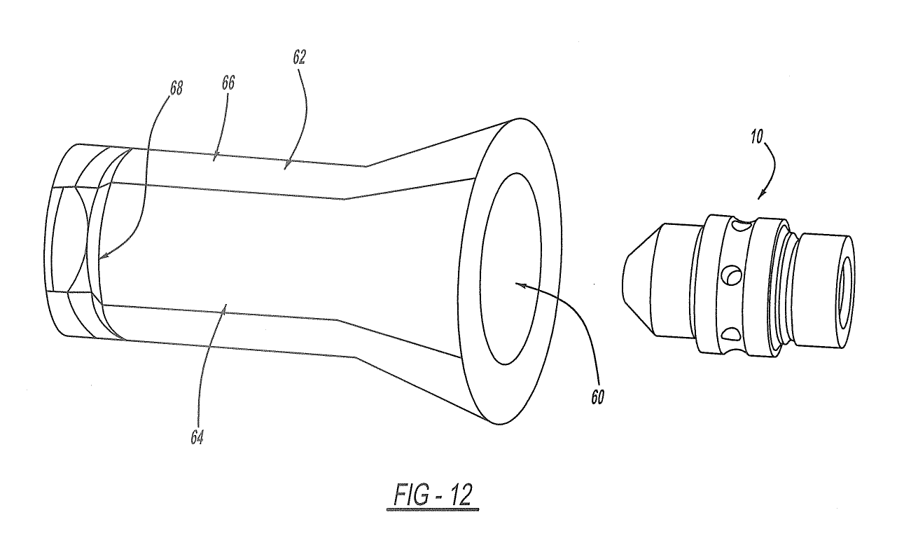

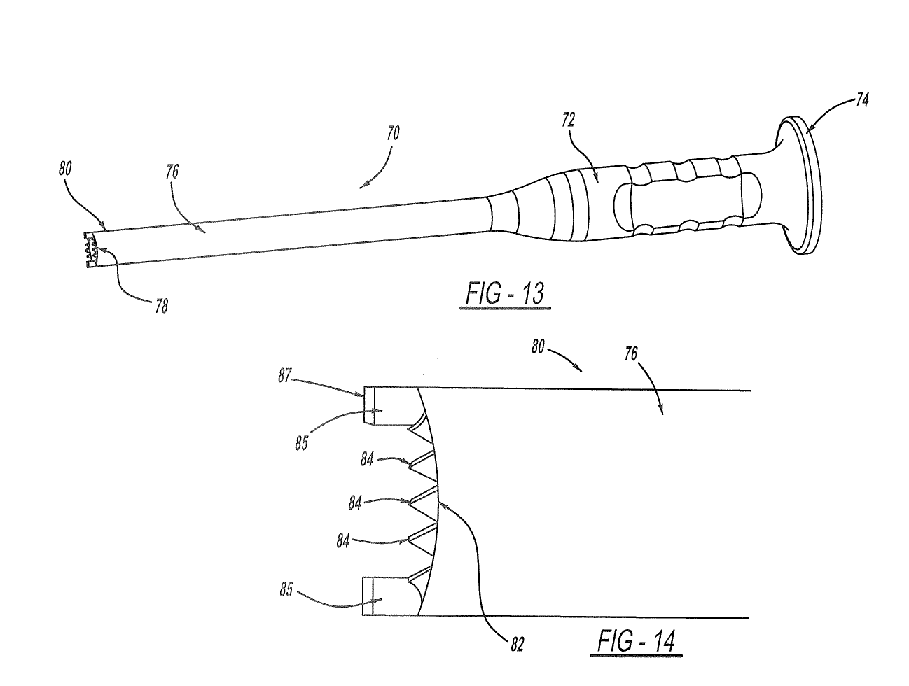

More specifically, the implant 10 can be inserted into the facet joint through the use of a guide instrument 70, shown in FIG. 13. The guide Instrument 70 attaches to the bone surrounding the facet joint. The guide instrument 70 includes a handle portion 72 with an enlarged proximal end 74, a tubular portion 76 that is operably connected to and extends from the handle portion 72, and a bone engagement surface 78 at a distal end 80 of the tubular portion 76. The bone engagement surface 78 is detailed in FIG. 14. The bone engagement surface 78 includes an arc 82, which is drawn as a reference arc for purposes of clarification. Teeth 84 are formed along the arc 82, such that the base of the teeth 84 and the tips of the teeth 84 are situated along this arc 82 and are not on the same plane. Another way of forming the teeth 84 are by allowing their height to vary such that the base of the tooth 84 is not on an arc 82, but the tip of the teeth 84 are on the arc 82. This is for the simple reason that the bone features surrounding the facet joint are not flat, but curved. Thus, to provide better hold, the teeth 84 should be cut along an arc or curve to better match the anatomy. This allows the guide tube 70 to be held in position with much less force than one having the teeth be cut such that the tips are all in the same plane. Teeth 85 are teeth with a flat surface 87 designed to slide into the facet joint in the same plane as the joint.

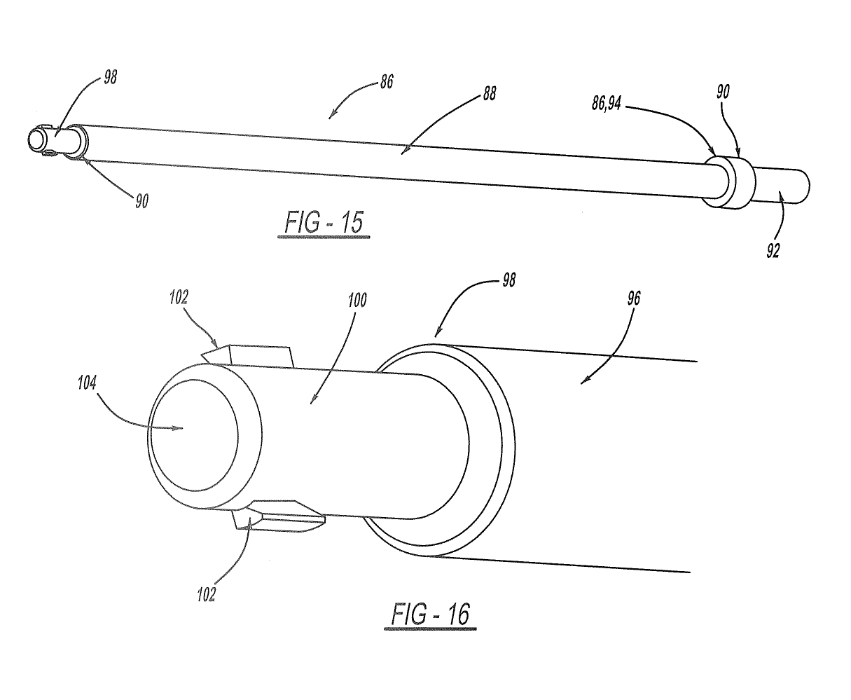

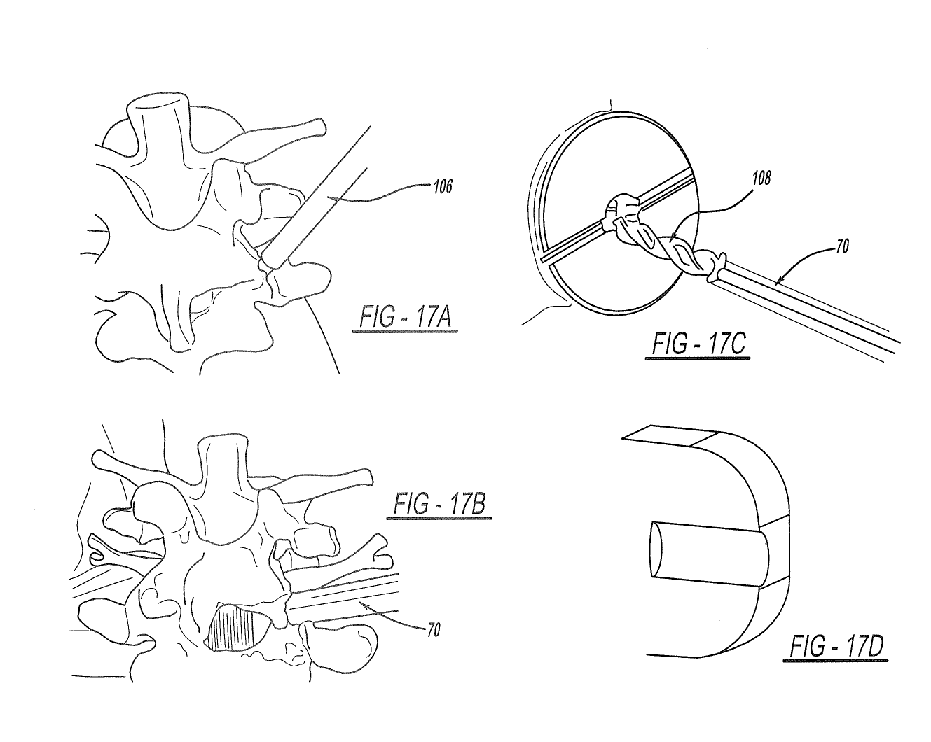

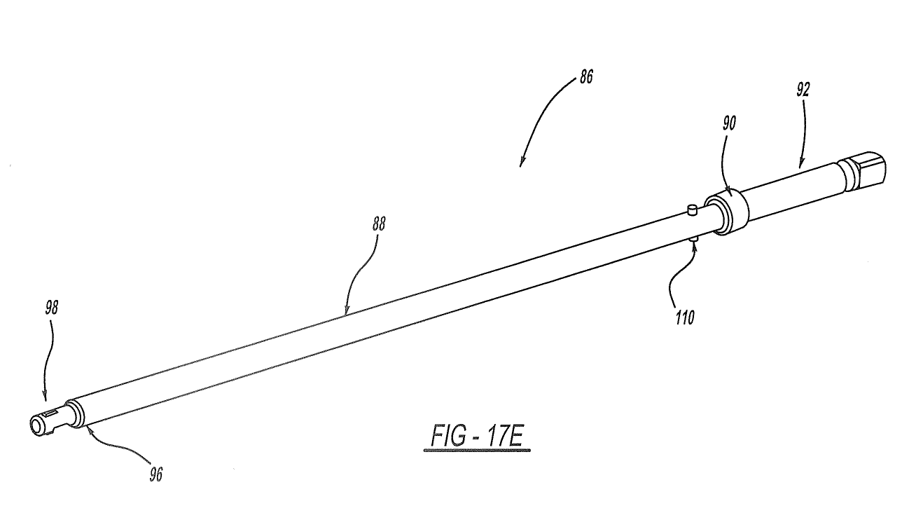

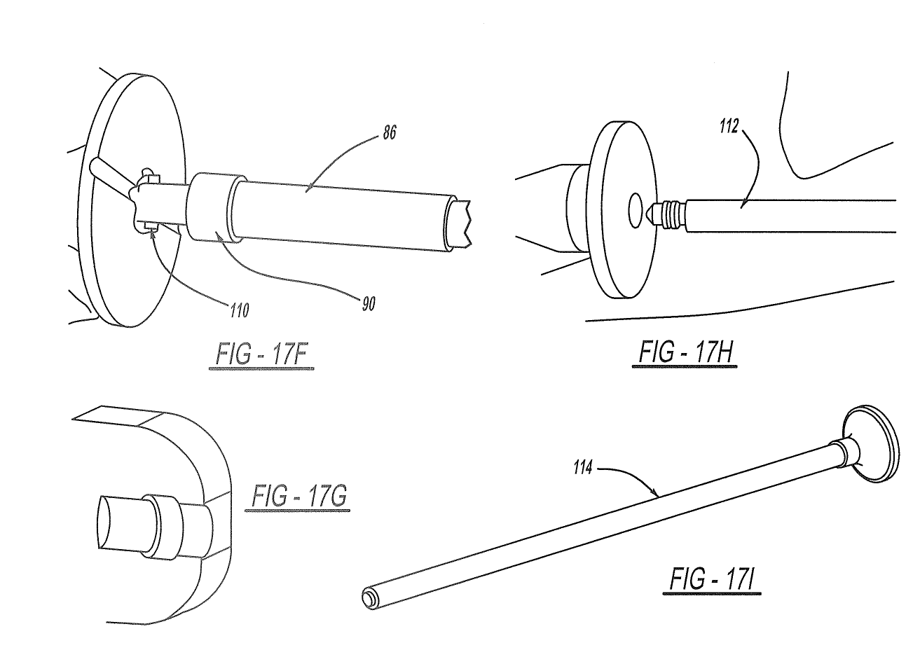

Another instrument that aids in the insertion of the implant 10 along with the guide instrument 70 is a reamer 86, shown in FIGS. 15 and 16. The reamer 86 includes a shaft 88 having a collar 90 at a proximal end 92, the collar including a face 94 acts as a stop against the enlarged proximal end 74 of the guide instrument 70. The shaft 88 can also include an alignment mechanism 110 for aligning the reamer 86 with the guide instrument 70, shown in FIG. 17E. The proximal end 92 is designed to interface with a handle for turning the shaft 88. The proximal end 92 can have a square drive, AO drive, or have other attachment mechanisms to accept a handle. The distal end 96 includes a cutting mechanism 98 with a cutting shaft 100 that is smaller than the diameter of the facet fusion implant 10 to allow for clearance. Cutting teeth 102 on cutting shaft 100 are generally reaming teeth constructed with the appropriate angles and form to best cut bone. The area between the end of the teeth 102, and a distal end 104 of the cutting shaft 100, is cylindrical and acts along with the cutting shaft 100 as a guide to help maintain concentricity of the reamed cut. The cutting mechanism 98 is turned at least 180 degrees in order to cut the full 360 degree arc required to fit the facet fusion implants 10 such as shown in FIGS. 6 and 7. Of course, for facet fusion implants as shown in FIGS. 1, 2, and 3, only a partially cut arc is required.

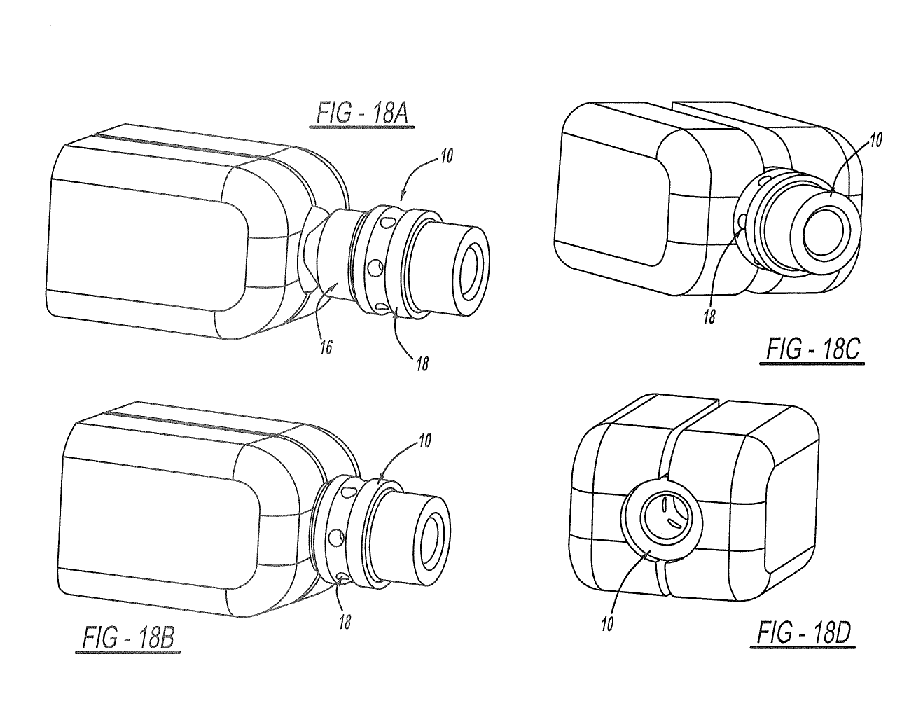

The guide instrument 70 and the reamer 86 are used as follows in order to prepare the facet joint for insertion of the implant 10 and as illustrated in FIGS. 17A-17I. First, a facet locator 106 having a flat tip is used to locate the center of the facet and the relative angle of the joint (FIG. 17A). The tip of the facet locator 106 is inserted into the joint space. For minimally invasive access, a K-wire, which is a small diameter wire or pin, can be inserted via X-ray guidance from a C-ARM and the facet locator 106 slid over the pin. The guide instrument 70 is then inserted over the facet locator 106 until the distal end 80 contacts the bone around the facet joint (FIG. 17B). The facet locator 106 can also include a groove to aid in aligning the guide instrument 70. The guide instrument 70 can then be held against the bone or tapped into place and the locator is then removed. A drill 108 matched to the smaller diameter of the implant 10, such as the first section 16 in the Figures, is then inserted into the guide and preferably a hole the size of the smaller diameter is drilled to the proper depth centered in the facet joint (FIG. 17C). The depth of the hole is controlled by a stop on the drill 108. Teeth 84, 85 of the guide instrument 70 are in alignment with the facet joint. Some interference of the implant 10 with the hole is allowed for to permit solid contact of the implant 10 with the facet joint bone. The drill 108 prepares a round hole; however, there are two sides, each side of the joint has slightly less than half of the formed hole (FIG. 17D). The drill 108 is then removed and the reamer 86 (such as in FIG. 17E) inserted until it the face 94 engages the enlarged proximal end 74 of the guide instrument 70 to act as a stop in order to position the reamer 86 at the correct depth. The reamer alignment mechanism 110 only allows the reamer 86 to enter the facet joint if aligned properly with the guide instrument 70, and the reamer 86 can only be extracted after cutting if the reamer 86 is aligned properly (FIG. 17F). This helps guarantee that the pocket is formed and not cut away when the reamer 86 is removed. The reamer 86 is turned by hand and cuts a pocket within the facet joint for the larger section of the facet fusion implant 10 (FIG. 17G), with the pocket placed such that the posterior aspect of the pocket is within the facet joint bone. In effect, this creates the negative of the implant 10, whereby the reamed out section or pocket matches the larger diameter section (such as second section 18) of the implant 10 and the pocket is away from the drilled opening. The reamer 86 is then removed by lining the distal end 96 parallel to the facet joint. With the cavity for the implant 10 created, the implant 10 can be introduced into the machined space with an inserter 112 that is introduced into the guide instrument 70 and the implant 10 is seated into the facet joint until the inserter 112 contacts the guide instrument 70 (FIG. 17H). The implant 10 is first positioned at the prepared opening in the facet joint as shown in FIG. 18A. FIG. 18B shows the implant 10 with the smaller diameter section (first section 16) in the facet joint opening. As the implant 10 is pushed in further, the larger diameter section (second section 18) of the implant 10 forces the facet joint to open and the gap between the facets increases (FIG. 18C). The inserter 112 can be loosened and removed, leaving the implant 10 behind. At this point, the implant 10 is within the facet joint but not fully seated. Next, a counter sink 114 (FIG. 171) is introduced into the guide instrument 70 until it contacts the base of the implant 10. Light pressure or taps can be exerted against the counter sink 114, which forces the larger diameter section of the implant 10 to seat within the reamed pocket. The pocket that has been created fits the implant and the implant can simply pop right in place with minimal tapping. As the facet joint is still somewhat mobile during insertion, the larger diameter section of the implant 10 can be introduced without damage to the implant 10 or facet joint, as the joint will move slightly to accept the implant 10. In addition, for dehydrated bone materials, bone is smaller when dry and grows when rehydrated. Thus, it is possible to insert the implant 10 when the bone is not completely rehydrated to make the insertion of the implant 10 easier, should this be needed. The facet joint closes around the implant 10 and this compressive force securely locks the facet implant in place (FIG. 18D). The implant 10 is counter sunk relative to the facet joint face. Due to variations in facet joint geometry and osteophytes, this allows the instrumentation to compensate and allow the implant 10 to seat correctly. The guide instrument 70 is then removed.

The present invention also provides generally for a method of inserting an implant into a facet joint, by creating a pocket within the facet joint that is a negative image of the implant, and popping the implant in place within the pocket. Each of these steps have been described above.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A facet fusion implant, comprising a body including stepped surface means for contacting a stepped surface of a facet joint and preventing said facet fusion implant from coming out of said facet joint.

2. The facet fusion implant of claim 1, wherein said body is a shape chosen from the group consisting of cylindrical, square, and rectangular.

3. The facet fusion implant of claim 1, wherein said body is made of a material chosen from the group consisting of autogenous bone, allograft bone, bone substitutes, polymers, and porous materials.

4. The facet fusion implant of claim 1, wherein said stepped surface means is further defined as at least two sections on said body that are different sizes in diameter.

5. The facet fusion implant of claim 4, wherein said stepped surface means is further defined as a first section with a smaller diameter operably connected to a second section having a larger diameter than said first section.

6. The facet fusion implant of claim 5, further including a third section operably connected to said second section and having a diameter the same size as said first section.

7. The facet fusion implant of claim 6, wherein said second section includes at least one machined away portion including a flat surface.

8. The facet fusion implant of claim 6, wherein said third section includes flat surface means for engaging with an instrument.

9. The facet fusion implant of claim 6, wherein said stepped surface means is machined from a single piece of material.

10. The facet fusion implant of claim 5, wherein said second section is further defined as sleeve means for slipping over said first section and creating said stepped surface means.

11. The facet fusion implant of claim 5, wherein said second section further includes a face that is 90 degrees relative to said first section.

12. The facet fusion implant of claim 5, further including a chamfered transition between said first section and said second section.

13. The facet fusion implant of claim 5, wherein said first section includes a tip having a tip chamfer.

14. The facet fusion implant of claim 13, wherein said tip includes a flat surface.

15. The facet fusion implant of claim 14, further including a small blend radius between said flat tip and said tip chamfer.

16. The facet fusion implant of claim 6, said body further including groove means for increasing surface grip and carrying bone enhancing materials.

17. The facet fusion implant of claim 16, wherein said second section includes a central groove.

18. The facet fusion implant of claim 17, wherein said first and third sections each include at least one groove.

19. The facet fusion implant of claim 16, wherein said groove means includes therein bone enhancing materials chosen from the group consisting of Demineralized Bone Matrix (DBM), calcium sulfate, BMP's (Bone Morphogenetic Proteins), autograft, and stem cell containing materials or carriers.

20. The facet fusion implant of claim 6, said body further including internal bore means for carrying bone enhancing materials.

21. The facet fusion implant of claim 20, wherein said internal bore means runs a partial length of said body.

22. The facet fusion implant of claim 20, wherein said internal bore means runs the entire length of said body.

23. The facet fusion implant of claim 22, wherein said internal bore means includes a chamfer at a proximal end.

24. The facet fusion implant of claim 22, wherein said internal bore means includes therein bone enhancing materials chosen from the group consisting of Demineralized Bone Matrix (DBM), calcium sulfate, BMP's (Bone Morphogenetic Proteins), autograft, and stem cell containing materials or carriers.

25. The facet fusion implant of claim 22, said body further including at least one outer hole means for flowing blood and bone enhancing materials to said internal bore means.

26. The facet fusion implant of claim 25, wherein said outer hole means is further defined as an array of multiple holes on said body in fluid connection with said internal bore means.

27. The facet fusion implant of claim 25, wherein said body is made of a naturally porous material and wherein said outer hole means are integral in said material.

28. The facet fusion implant of claim 27, wherein said naturally porous material is chosen from the group consisting of titanium foam and tantalum foam.

29. The facet fusion implant of claim 25, wherein said outer hole means are at an angle chosen from the group consisting of perpendicular to said internal bore and angled relative to an axial centerline of said implant.

30. A facet fusion implant comprising a body including stepped surface means for contacting a stepped surface of a facet joint and preventing said facet fusion implant from coming out of said facet joint, said body also including groove means for increasing surface grip and carrying bone enhancing materials.

31. A facet fusion implant comprising a body including stepped surface means for contacting a stepped surface of a facet joint and preventing said facet fusion implant from coming out of said facet joint, and said body including internal bore means for carrying bone enhancing materials.

32. A facet fusion implant comprising a body including stepped surface means for contacting a stepped surface of a facet joint and preventing said facet fusion implant from coming out of said facet joint, said body including internal bore means for carrying bone enhancing materials, and at least one outer hole means for flowing blood and bone enhancing materials to said internal bore means.

33. An external sleeve compacting instrument for inserting bone enhancing materials in an implant, comprising a tube including at least one flexible portion and an opening section that expands outward.

34. The external sleeve compacting instrument of claim 33, wherein said opening section is in the shape of a funnel.

35. The external sleeve compacting instrument of claim 33, wherein the diameter of said opening section is larger than the diameter of the implant.

36. The external sleeve compacting instrument of claim 33, further including bone enhancing materials within said tube.

37. The external sleeve compacting instrument of claim 33, wherein said flexible portion is made out silicone.

38. The external sleeve compacting instrument of claim 33, wherein said external sleeve compacting instrument is disposable.

39. The external sleeve compacting instrument of claim 33, wherein said tube further includes an orifice at an end opposite said opening section.

40. A method of using an external sleeve compacting instrument, including the steps of:
inserting bone enhancing materials through an opening section that expands outward, and into a tube;
pushing an implant through the opening section and into the tube; and
forcing the bone enhancing material into a structure of the implant chosen from the group consisting of grooves, internal bores, outer holes, rough body surfaces, and combinations thereof by a flexible portion of the tube.

41. The method of claim 40, further including the step of pushing the implant through an orifice at an end opposite the opening section and removing the implant from the external sleeve compacting instrument.

42. A method of preparing a facet fusion implant with bone enhancing materials, including the step of:
inserting bone enhancing material into an internal bore of a body of the implant, the body including stepped surface means for contacting a stepped surface of a facet joint and preventing said facet fusion implant from coming out of said facet joint.

43. The method of claim 42, wherein said inserting step is further defined as inserting a plug of bone enhancing materials into the internal bore.

44. The method of claim 42, wherein said inserting step is further defined as inserting a syringe into the internal bore, injecting bone enhancing materials into the internal bore, and forcing the bone enhancing materials through outer holes on the body in fluid connection with the internal bore.

45. The method of claim 42, wherein said inserting step is further defined as inserting bone enhancing materials through an opening section of an external sleeve compacting instrument that expands outward, and into a tube;
pushing the implant through the opening section and into the tube; and
forcing the bone enhancing material into a structure of the implant chosen from the group consisting of grooves, internal bores, outer holes, rough body surfaces, and combinations thereof by a flexible portion of the tube.

46. The method of claim 45, further including the step of pushing the implant through an orifice at an end opposite the opening section and removing the implant from the external sleeve compacting instrument.

47. A method of creating a facet joint fusion, including the steps of:
inserting an implant in a facet joint, the implant including a body having a stepped surface;
contacting the stepped surface with a stepped surface of the facet joint;
preventing motion of the implant within the facet joint; and
creating a facet joint fusion.

48. The method of claim 47, wherein said inserting step is further defined as locating a center of the facet joint with a facet locator, inserting a guide instrument over the facet locator until a distal end of the guide instrument contacts bone around the facet joint, removing the facet locator, drilling a hole in the facet joint matching a first diameter of the stepped surface of the implant, cutting a pocket in the facet joint with a reamer matching a second diameter of the stepped surface of the implant, tapping the implant into the facet joint, and removing the guide instrument.

49. The method of claim 47, further including the step of inserting bone enhancing material into the implant prior to said inserting in a facet joint step by a method chosen from the group consisting of inserting a plug, injecting bone enhancing material with a syringe, and using the external sleeve compacting instrument of claim 33.

50. The method of claim 47, wherein said contacting step is further defined as contacting a first section having a small diameter and a second section having a larger diameter than the first section of the implant with the stepped surface of the facet joint.

51. The method of claim 47, wherein said preventing step is further defined as preventing posterior and anterior motion and allowing the implant to completely fuse with the facet joint.

52. A method of preventing motion of an implant within a facet joint, including the steps of:
contacting a stepped surface of the implant with a stepped surface of the facet joint; and
preventing motion of the implant within the facet joint.

53. A guide instrument for inserting an implant in a facet joint, comprising a handle with an enlarged proximal end, a tubular portion operably connected to said handle, said tubular portion including a distal end having bone engagement means for attaching to bone surrounding the facet joint.

54. The guide instrument of claim 53, wherein said bone engagement means is further defined as teeth means along an arc for attaching to bone surrounding the facet joint.

55. The guide instrument of claim 54, wherein said teeth means are not on the same plane.

56. The guide instrument of claim 54, wherein said teeth means are further defined as teeth with varying height of tips along the arc.

57. A reamer for preparing a cavity and step for an implant in a facet joint, comprising a shaft including a proximal end, said proximal end having stop means for stopping against a guide instrument and interface means for interfacing with a handle, and a distal end including cutting means for cutting bone in said facet joint.

58. The reamer of claim 57, wherein said stop means is further defined as a collar including a face.

59. The reamer of claim 57, wherein said cutting means is further defined as a cutting shaft including cutting teeth.

60. The reamer of claim 57, further including a cylindrical guide portion between said cutting teeth and a distal end of said cutting shaft.

61. The reamer of claim 57, further including alignment means for aligning said reamer within a guide instrument.

62. A method of inserting an implant into a facet joint, including the steps of:
locating the center of the facet joint and the relative angle with a facet locator;
inserting the guide instrument of claim 53 over the facet locator until the distal end contacts the bone around the facet joint;
removing the facet locator;
drilling a hole that matches a first diameter of the implant;
inserting the reamer of claim 57 until the stop means engages the enlarged proximal end of the guide instrument;
cutting a pocket with the reamer within the facet joint bone matching a
second diameter of the implant;
removing the reamer;
inserting the implant in the facet joint; and
removing the guide instrument.

63. The method of claim 62, wherein said locating step further includes the step of inserting a K-wire via X-ray guidance from a C-ARM and the facet locator slid over the K-wire.

64. The method of claim 62, wherein said cutting a pocket step creates a negative of the implant.

65. The method of claim 62, wherein said inserting the implant step is further defined as tapping the implant until a section having the second diameter is disposed within the reamed pocket.

66. The method of claim 65, wherein said tapping step is performed with an inserter.

67. A method of inserting an implant into a facet joint, including the steps of:
creating a pocket within the facet joint that is a negative image of the implant;
and popping the implant in place within the pocket.

68. A facet fusion implant comprising a body including locking means for locking said body in a facet joint.

69. The facet fusion implant of claim 68, wherein said locking means are further defined as anterior force applying means for applying an anterior force to a drilled portion of the facet joint and posterior force applying means for applying a posterior force in the facet joint.

70. A method of locking an implant in a facet joint, including the steps of:
applying force to the implant in an anterior direction;
applying force to the implant in a posterior direction; and
locking the implant in the facet joint.

71. The method of claim 70, wherein said applying force to the implant in an anterior direction step is further defined as preventing anterior movement of the implant due to drill depth.

72. The method of claim 71, wherein said applying force to the implant in a posterior direction step is further defined as preventing posterior movement of the implant by a stepped surface of the implant.
